(19) United States
(12) Patent Application Publication
(10) Pub. No.: US 2004/0192683 A1
(43) Pub. Date: Sep. 30, 2004

(54) Active ingredient combination for treating a dependence on addictive substances or narcotics using medicaments

(76) Inventors: Joachim Moormann, Werne (DE); Hermann Mucke, Osterreich (DE); Klaus Opitz, Munster (DE)

Correspondence Address: BIRCH STEWART KOLASCH & BIRCH, PO BOX 747, FALLS CHURCH, VA, 22040-0747, US

(21) Appl. No.: 10/480,287

(22) PCT Filed: Jun. 15, 2002
(86) PCT No.: PCT/EP02/06630

(30) Foreign Application Priority Data
Jun. 18, 2001 (DE) 101 29 265.1

(51) Int. Cl. A61K 31/54; A61K 31/4015

(52) U.S. Cl. 514/228.2; 514/424

(57) ABSTRACT

The present invention relates to an active ingredient combination composed of at least one modulator of the cholinergic system with at least one substance having antiexcitatory activity for pharmacological addictive substance or intoxicant therapy, in particular of alcoholism.

The present invention relates to active ingredient combinations and to the use thereof for pharmacological addictive substance or intoxicant therapy, especially relating to alcohol. In this connection, the active ingredient combination consists of at least one modulator of the cholinergic system with at least one substance with antiexcitatory activity. The present invention further relates to the use of the said active ingredient combination for producing medicaments which contribute to the therapy of the consumption of addictive substances or intoxicants, in particular the consumption of alcohol

Intake of addictive substances and intoxicants, especially alcohol, is well known to lead to symptoms such as perception disturbances, memory loss, impairment of cognitive abilities, general loss of control, aggressiveness, impairment of muscular co-ordination, etc. If the intoxicant is deliberately taken, then although such effects are intended by the intoxicant-consuming person, they are also under certain conditions felt to be disadvantageous. An additional factor is that the severity and the duration of these symptoms may vary and is often difficult for the consumer of the intoxicant to estimate beforehand.

Especially when there is chronic dependence and continued abuse of intoxicants there is not only the generally known organic damage but there is also the occurrence of permanent defunctionalization manifestations which impair, for example, cognitive performance, especially memory performance. This may also lead to sporadic or permanent dementing states. There may also be chronic manifestations of the previously mentioned psychiatric symptoms such as, for example, a general loss of control. These chronic sequelae of alcohol abuse—which occur in a similar way in other intoxicant dependencies—represent a considerable impediment to successful implementation of detoxification therapies. Thus, it is known that the loss of control caused by chronic alcohol abuse makes abstinence impossible for the person affected by alcoholism. This is the main reason why even detoxified alcoholics are prone to relapses, usually with serious consequences. The principle that “controlled drinking” is impossible for dependent people was derived from this observation.

It is additionally known that there are great individual differences in intoxicant consumption behaviour, which is why, for example, alcoholics are divided into different categories of drinkers.

The problem for certain alcohol consumers is that, after a particular individual threshold dose has been exceeded, there is a rapid general loss of control with the abovementioned adverse side effects. The affected persons are usually unable to recognize in good time that they have reached their individual threshold dose or even their personal risk of relapse. The loss of control brought about thereby often leads to further excessive alcohol consumption. These are frequently people who have already undergone withdrawal therapies and relapse in this way.

It is known that the loss of control caused by chronic abuse of addictive substances, as well as the impairment of memory performance (and even dementia), often has far-reaching consequences for the affected person and for his surroundings, such as, for example, inability to carry on an occupation, inability to organize daily activities, inability to initiate and maintain social contacts and, resulting therefrom, social isolation.

The addictive substance-related defunctionalization manifestations, e.g. the impairment of cognitive performance, often persist even after successfully completed withdrawal therapy. Further psychiatric or cerebral disturbances occurring in association with alcohol abuse or abuse of other addictive substances are, for example: perceptual illusions or hallucinations, amnesia, alterations of consciousness, formal cognitive disturbances, memory deficits, delusions, confabulations, disorientation, states of agitation.

At present, five products are approved in European countries and/or in the United States of America for pharmacological therapy of alcohol abuse. The one which has been used longest is bis (diethylthiocarbamoyl) disulphide (disulfiram, Antabuse®), which leads, through blocking of aldehyde dehydrogenase, to an accumulation of toxic end products of alcohol breakdown and, consequently, to nausea after alcohol consumption. Despite the aversive effect, the actual desire for alcohol is unaffected. Tiapride, a dopamine antagonist which acts on dopamine receptor subtypes D2 and D3 has achieved scarcely any practical importance. Used to a far larger extent are the opiate receptor antagonist naltrexone (ReVia®, DuPont, Trexan®) and acamprosate (Campral®, Merck AG; Aortal®), which acts in a complex manner, to prevent relapses in alcohol abuse after successful alcohol detoxification. Gamma-hydroxybutyrate (for example Alcover®, Gerot Pharmazeutika) has recently become available in a few European countries. However, naltrexone and gamma-hydroxybutyrate cause considerable gastrointestinal and psychomotor side effects which impair compliance with the therapy. Naltrexone is moreover characterized by a low oral bioavailability and, in addition, is hepatotoxic, whereas gamma-hydroxybutyrate itself has addictive potential.

Nevertheless, the long-term successes of all the approved drugs must overall be designated very limited because, in the majority of patients, they bring about only marginal delays in relapse after detoxification or only a clinically insignificant reduction in the quantity of alcohol. The fact that on average only about 30% of all patients are still abstinent one year after detoxification treatment has not been permanently affected by these medicaments. In addition, therapy of the early stages of the development, which often extends over decades, of alcoholism requires medicaments with particularly few side effects, because the so-called social drinkers have, owing to the level of suffering still being low, scarcely any insight into the problems of their drinking behaviour and therefore are not very prepared to put up with the side effects of pharmacological alcohol therapy. There has thus been no lack of attempts for many years to introduce pharmacological improvements into alcohol therapy, although the proposed substances and substance combinations, and the use thereof in the therapy of alcoholism, were, with few exceptions, already known previously. The combined use of acamprosate and naltrexone which is described in EP 0 945 133 is an example of such a combination of active ingredients, each of which has been used for some years in the therapy of alcohol abuse. However, according to very recent studies (Neurosci. Behav. 2001; 23(2): 109-118), no synergistic effect is to be ascribed to this combination. The publications DE 40 10 079 and U.S. Pat. No. 5,519,017 propose, as alternative for the treatment of alcohol abuse, the use of galanthamine which is said to suppress the desire for nicotine and alcohol. In addition, U.S. Pat. No. 5,932,238 describes a transdermal therapeutic system suitable for galanthamine.

Galanthamine is also used for the treatment of poliomyelitis, of Alzheimer's disease and of various disorders of the nervous system, and for the treatment of closed angle glaucoma.

Galanthamine or galantamine (4a,5,9,10,11,12-Hexahydro-3-methoxy-11-methyl-6-H-benzofuro-(3a,3,2-ef)-(2)-benzazepin-6-ol) is a tetracyclic alkaloid which occurs in certain plants, especially in amaryllidaceae. It can be isolated from these plants by known processes (for example as disclosed in DE 195 09 663 A1 or DE-PS 11 93 061) or by a synthetic route (for example Kametani et al., Chem. Soc. C. 6, 1043-1047 (1971) or Shimizu et al., Heterocycles 8, 277-282 (1977)).

On the basis of its pharmacological properties, galanthamine is included in the group of reversibly acting cholinesterase inhibitors. At the same time, galanthamine also stimulates the release of the neurotransmitter acetylcholine through direct stimulation of the presynaptic nicotinic acetylcholine receptors. An analogous process also takes place at dopaminergic presynaptic nerve endings, where it promotes the release of dopamine. These properties of galanthamine are said according to current theories to reduce the craving for alcohol independently of cognitive control, which forms the theoretical basis for the publications DE-40 10 079 and U.S. Pat. No. 5,932,238.

The combined direct cholinergic and indirect dopaminergic effect described for galanthamine can also be achieved with substances which simultaneously inhibit acetylcholinesterase and monoamine oxidase. This is the case for example with deoxypeganine which is also referred to as deoxyvasicine, especially in the older literature. For this reason, DE 199 06 974 also claims deoxypeganine for the therapy of alcohol abuse. It was additionally proposed to use deoxypeganine likewise for the pharmacological therapy of Alzheimer's dementia, for the treatment of nicotine dependence through reducing the desire for nicotine or for replacement therapy of drug addicts and for the treatment of withdrawal symptoms during withdrawal therapy. In addition, deoxypeganine can, as cholinesterase inhibitor be employed as antidote or prophylactic in cases of poisoning by organic phosphates, in which case it antagonizes the cerebral effect of cholinergic poisons.

Deoxypeganine (1,2,3,9-tetrahydropyrrolo[2,1-b]quinazoline)is an alkaloid of molecular formula C₁₁H₁₂N₂ which is present in plants of the zygophyllaceae family. Deoxypeganine is preferably obtained by isolation from Syrian rue (Peganum harmala) or by synthesis.

Despite their duplicated mechanisms of action, galanthamine and deoxypeganine have only restricted suitability for effective suppression of the desire for addictive substances or intoxicants. The reason for this is likely to be that the desire for alcohol is, according to the current state of knowledge, essentially caused in part by neuronal over-excitation. This overexcitation drives the dependent person repeatedly to new drinking because the acute intoxication with alcohol depresses this overexcitation of the nervous system. Neither galanthamine nor deoxypeganine influence the chronic neuronal overexcitation, so that suppression of the craving is not possible by these substances on their own.

The aim of the present invention was therefore to provide medicaments through which the alcohol-induced excitation is depressed without, however, impairing to a relevant extent the physiological excitatory stimulus conduction, so that the medicaments obtained in this way have no unreasonable side effects such as, for example, strong sedation or impairment of cognition, in order to reduce alcohol consumption.

It has surprisingly been found that the object on which the present invention is based can be solved particularly well by the combination of a modulator of the cholinergic system with substances having antiexcitatory activity from particular subgroups.

It is possible to use according to the invention modulators of the cholinergic system which, besides their inhibitory effect on cholinesterases, also act on dopaminergic nerve endings. This is possible for example with substances which, as cholinesterase inhibitors, also directly stimulate nicotinic acetylcholine receptors at the presynaptic nerve endings of cholinergic and dopaminergic nerve endings, or with substances which simultaneously inhibit acetylcholin-esterase and monoamine oxidase.

The modulators of the cholinergic system having the properties mentioned above which are preferably used are galanthamine or deoxypeganine or pharmacologically acceptable derivatives thereof. It is self-evident to the skilled person that galanthamine or deoxypeganine are used in the form of their free bases or in the form of their known salts or derivatives. Thus, for example, in place of the salts or addition compounds of galanthamine it is also possible to use all galanthamine derivatives mentioned or claimed in the scientific literature and in patents as long as they are either inhibitors of cholinesterase enzymes or modulators of nicotinic acetylcholine receptors, or combine both pharmacological activities. These include, in particular:

The compounds mentioned in the patents of the families WO-9612692/EP-0787115/U.S. Pat. No. 6043359 and WO-9740049/EP-0897387 and WO-032199 (Waldheim Pharmazeutika GmbH. and Sanochemia Pharmazeutika AG), including, in particular:

(−)-N-Demethylgalanthamine;

(−)-(N-Demethyl)-N-allylgalanthamine;

(−)-(6-Demethoxy)-6-hydroxygalanthamine (SPH-1088);

(+/−) N-Demethylgalanthamine N-tert-butyl carboxamide (SPH-1221);

(−) N-Demethylgalanthamine N-tert-butyl carboxamide

The compounds mentioned in the patents of the families EP-0648771 and EP-0653427 (Hoechst Roussel Pharmaceuticals Inc.) and Drugs Fut. 21(6), 621-635 (1996) and J. Pharmacol. Exp. Ther. 277(2), 728-738 (1996), including, in particular:

(−)-6-O-Demethylgalanthamine;

(−)-(6-O-Acetyl)-6-O-demethylgalanthamine (P11012);

(−)-(6-O-Demethyl)-6-O-[(adamantan-1-yl)carbonyl] galanthamine (P11149);

(−)-(6-O-Demethyl)-6-O-(triethylsilyl)galanthamine;

(−)-(6-O-Demethyl)-6-O-(triisopropylsilyl)galanthamine;

(−)-(6-O-Demethyl)-6-O-(trimethylsilyl)galanthamine;

The compounds mentioned in the patents of the families WO-9703987/EP-0839149/U.S. Pat. No. 5958903 (Societe de Conseils de Recherches et D'Applications Scientifiques, S.C.R.A.S) including, in particular:

(6-O-Demethyl)-6-O-(8′-phthalimidooctyl)galanthaminium bromohydrate;

(6-O-Demethyl)-6-O-(4′-phthalimidobutyl)galanthaminium bromohydrate;

(6-O-Demethyl)-6-O-(10′-phthalimidodecyl)galanthaminium bromohydrate;

(6-O-Demethyl)-6-O-(12′-phthalimidododecyl)galanthaminium bromohydrate;

10-N-Demethyl-10-N-(10′-phthalimidobutyl)galanthaminium trifluoroacetate;

10-N-Demethyl-10-N-(10′-phthalimidohexyl)galanthaminium trifluoroacetate;

10-N-Demethyl-10-N-(10′-phthalimidooctyl)galanthaminium bromohydrate;

10-N-Demethyl-10-N-(10′-phthalimidododecyl)galanthaminium bromohydrate;

10-N-Demethyl-10-N-(12′-phthalimidododecyl)galanthaminium bromohydrate;

10-N-Demethyl-10-N-(6′-pyrrolohexyl)galanthaminium bromohydrate

The (−)N,N′-demethyl-N,N′-bisgalantamine derivatives, which are described inter alia in the publication Bioorg. Med. Chem. 6(10), 1835-1850 (1998), of the following structural formula, where the bridging group (“balkyl spacer”) between the nitrogen atoms of the two galanthamine molecules may be 3-10 CH₂ groups long and, independently thereof, one of the two galanthamine molecules may carry a positive charge on the nitrogen atom (galanthaminium cation):

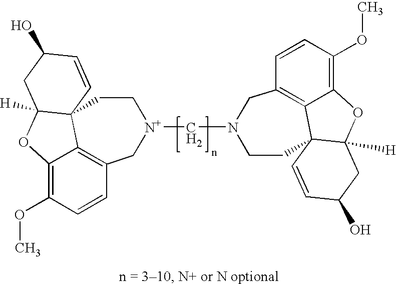

The (−)N-demethyl-N-(3-piperidinopropyl)galanthamine (SPH-1286), which is described inter alia in the publication J. Cerebral Blood Flow Metab. 19(Suppl. 1), S19 (1999) and in Proteins 42, 182-191 (2001), and its analogues with alkyl spacers up to 10 CH₂ groups long:

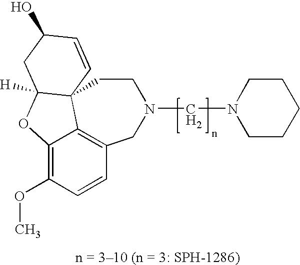

In place of deoxypeganine, its derivatives described in the literature are also to be understood in a similar way as long as they are simultaneously inhibitors of acetylcholinesterase and of monoamine oxidases. These include the 7-bromodeoxy-peganine described in Synthetic Communs. 25(4), 569-572 (1995), as well as the 7-halo-6-hydroxy-5-methoxydeoxy-peganines which are described in Drug Des. Disc. 14, 1-14 (1996) and have the general formula

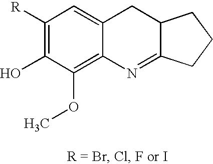

7-Bromo-6-hydroxy-5-methoxydeoxypeganine

7-Chloro-6-hydroxy-5-methoxydeoxypeganine

7-Fluoro-6-hydroxy-5-methoxydeoxypeganine

7-Iodo-6-hydroxy-5 -methoxydeoxypeganine

The deoxypeganine derivatives described in Ind. J. Chem. 24B, 789-790 (1985) can also furthermore be used.

The administered single dose of galanthamine or one of its pharmacologically acceptable salts or derivatives is preferably in the range from 1 to 50 mg, whereas the administered single dose of deoxypeganine or one of its pharmacologically acceptable salts or derivatives is preferably in the range from 10 to 500 mg.

According to the invention, galanthamine or deoxypeganine or one of their pharmacologically acceptable salts or derivatives are combined with at least one substance having antiexcitatory activity.

The object is achieved particularly advantageously by a combination with representatives of particular subgroups of pharmaceutically acceptable compounds having antiexcitatory activity.

These include, in particular,

the state-selective, noncompetitive antagonists of the activated NMDA receptor including, in particular, the substances to be found in the class of adamantane derivatives (such as, for example, memantine) and certain aminoalkylcyclohexane derivatives, and

compounds which, besides an antagonism at NMDA receptors, also exert an enhancing effect on the central GABAurgic system and thus a further depressant effect on the central nervous system, which are to be understood to include compounds from the structural class of linear aliphatic sulphonic and amino sulphonic acids such as, for example, derivatives of taurine, especially acamprosate; and

compounds which modulate metabotropic glutamate receptors in such a way that neuronal overexcitation is depressed in the manner described above.

It is evident that in place of acamprosate it is possible to employ salts of the aminoalkanesulphonic acid derivatives which are structurally related thereto and have comparable pharmacological activity, especially all those claimed in WO-9937606 (Lipha S.A.), including, in particular, the magnesium salt of 3-(2-methylpropanoylamino)propanesulphonic acid. The same applies to derivatives of memantine, which are to be understood to include all adamantane derivatives which bind to the activated form of N-methyl-D-aspartate receptor and block the effects thereon of ligands having excitatory activity. These are, in particular, other 1-aminoadamantane derivatives such as amantadine, but also memantine analogues with the same pharmacological properties, such as, for example, 1-amino-1,3,3,5,5-pentamethylcyclohexane (MRZ 2/579).

In addition, in the pharmaceutical preparations, galanthamine or deoxypeganine or their pharmacologically acceptable salts or derivatives are combined together with antagonists of various classes of central metabotropic glutamate receptors (mGluR). Particularly suitable mGluR antagonists are the compounds, claimed in WO-0026198 and WO-0026199, 3,6-dihydro-3,5-dimethyl-6-(4-ethoxyphenyl)-2-(4-methanesulphonyl-aminophenylsulphonyl)-2H-1,2-oxazine and 2-(4-acetylamino-benzenesulphonyl)-3,6-dihydro-3,5-dimethyl-6-(4-methoxy-phenyl)-2H-1,2-oxazine; and the 3-(3-chlorobenzoylamino)-1-[2-(3-chlorophenyl)-ethyl]-3-methylpyrrolidine-2-thione claimed in WO-0069816, and its relatives mentioned in this document.

The administered single dose of acamprosate or one of its pharmacologically acceptable salts or derivatives is preferably in the range from 100 to 5 000 mg, whereas the administered single dose of memantine or one of its pharmacologically acceptable salts or derivatives is preferably in the range from 1 to 50 mg. The dosage of the antagonists of various classes of central metabotropic glutamate receptors may be between 0.1 mg and 100 mg per medicament unit.

The pharmaceutical forms which can be used according to the present invention for administering a combination of modulators of the cholinergic system with a substance having antiexcitatory activity or a modulator of metabotropic glutamate receptors may comprise one or more of the following additives:

antioxidants, synergists, stabilizers;

preservatives;

taste masking agents;

colours;

solvents, solubilizers;

surfactants (emulsifiers, solubilizers, wetting agents, antifoams);

agents affecting the viscosity and consistency, gel formers;

absorption promoters;

adsorbents, humectants, glidants;

agents affecting disintegration and dissolution, fillers (extenders), peptizers;

release-delaying agents.

This list is not definitive; the suitable physiologically acceptable substances are known to the skilled person.

A combination of modulators of the cholinergic system with a substance having antiexcitatory activity can be administered orally or parenterally. It is possible to use known dosage forms such as tablets, coated tablets or pastilles for oral administration. Also suitable are liquid or semiliquid dosage forms, in which case the agent is in the form of a solution or suspension. Solvents or suspending agents which can be used are water, aqueous media or pharmacologically acceptable oils (vegetable or mineral oils). The medicaments containing a combination of modulators of the cholinergic system with a substance having antiexcitatory activity are preferably formulated as depot medicaments which are able to deliver this agent to the body in a controlled manner over a prolonged period.

It is also possible according to the invention for a combination of modulators of the cholinergic system with a substance having antiexcitatory activity to be administered by the parenteral route. For this purpose it is particularly advantageous to use transdermal or transmucosal dosage forms for the administration according to the invention of a combination of modulators of the cholinergic system with a substance having antiexcitatory activity, in particular adhesive transdermal therapeutic systems (active ingredient plasters). These make it possible to deliver the agent in a controlled manner over a prolonged period via the skin to the patient to be treated.

A further advantage is that misuse is less easily possible with parenteral administration forms than with oral dosage forms. The predetermined active ingredient-release area and the predetermined release rate mean that overdosage by the patient can be substantially ruled out. In addition, transdermal dosage forms are very advantageous because of other properties, e.g. avoidance of the first-pass effect or a better, more uniform control of the blood level.

Such transdermal systems containing a combination of modulators of the cholinergic system with a substance having antiexcitatory activity normally have an active ingredient-containing, contact adhesive polymer matrix which is covered on the side remote from the skin by an active ingredient-impermeable backing, and whose adhesive, agent-delivering surface is covered before application by a detachable protective layer. The manufacture of such systems and the basic materials and excipients which can be used therefor are known in principle to the skilled person; for example, the assembly of such transdermal therapeutical systems is described in German patents DE 33 15 272 and DE 38 43 239 or in U.S. Pat. Nos. 4,769,028, 5,089,267, 3,742,951, 3,797,494, 3,996,934 and 4,031,894.

The combination, according to the invention, of a modulator of the cholinergic system with a substance having antiexcitatory activity can be used in the therapy of addictive substance and intoxicant abuse in order to reduce the consumption of the addictive substance or intoxicant.

The combination, according to the invention, of a modulator of the cholinergic system with a substance having antiexcitatory activity can be used to produce medicaments for the therapy of addictive substance and intoxicant abuse in order to reduce the consumption of the addictive substance or intoxicant, especially the consumption of alcohol.

The object of the invention is achieved in an illustrative manner as follows, it not being intended to restrict the scope of the invention by this illustrative list.

### EXAMPLE 1

Medicament to be administered orally or transdermally and containing 1 mg to 50 mg of galanthamine in the form of one of its pharmacologically acceptable salts, preferably in the form of its hydrobromide, or addition compounds and 100 mg to 5 000 mg of a pharmacologically acceptable salt of N-acetylhomotaurine, preferably the potassium salt, per single dose.

### EXAMPLE 2

Medicament to be administered orally or transdermally and containing 1 mg to 50 mg of galanthamine in the form of one of its pharmacologically acceptable salts, preferably in the form of its hydrobromide, or addition compounds and 1 mg to 50 mg of 1-amino-3,5-dimethyladamantane per single dose.

### EXAMPLE 3

Medicament to be administered orally or transdermally and containing 10 mg to 500 mg of deoxypeganine in the form of one of its pharmacologically acceptable salts, preferably in the form of its hydrochloride, or addition compounds and 100 mg to 5 000 mg of a pharmacologically acceptable salt of N-acetylhomotaurine, preferably the potassium salt, per single dose.

### EXAMPLE 4

Medicament to be administered orally or transdermally and containing 10 mg to 550 mg of deoxypeganine in the form of one of its pharmacologically acceptable salts, preferably in the form of its hydrochloride, or addition compounds and 1 mg to 50 mg of 1-amino-3,5-dimethyladamantane per single dose.

1. Active ingredient combination composed of at least one modulator of the cholinergic system with at least one substance having antiexcitatory activity for pharmacological addictive substance or intoxicant therapy, in particular the therapy of alcoholism, characterized in that the modulator or at least one of the modulators of the cholinergic system is an inhibitor of acetylcholinesterase which is preferably selected from the group comprising galanthamine and deoxypeganine and the pharmacologically acceptable salts and derivatives thereof.

2. Active ingredient combination according to claim 1, characterized in that the substance having antiexcitatory activity or at least one of the substances having antiexcitatory activity is selected from the group of NMDA receptor antagonists, preferably selected from the group which comprises acamprosate and memantine and the pharmacologically acceptable salts and derivatives thereof.

3. Active ingredient combination according to claim 1, characterized in that the substance having antiexcitatory activity is a modulator of metabotropic glutamate receptors which is preferably selected from the group which comprises 3,6-dihydro-3,5-dimethyl-6-(4-ethoxyphenyl)-2-(4-methane-sulphonylaminophenylsulphonyl)-2H-1,2-oxazine and 2-(4-acetylaminobenzenesulphonyl)-3,6-dihydro-3,5-dimethyl-6-(4-methoxyphenyl)-2H-1,2-oxazine; 3-(3-chlorobenzoylamino)-1-[2-(3-chlorophenyl)-ethyl]-3-methylpyrrolidine-2-thione and its pharmacologically acceptable derivatives.

4. Active ingredient combination according to any of claims 1 to 3, characterized in that it is in the form of a pharmaceutical form where the administered single dose of galanthamine or one of its pharmacologically acceptable salts or derivatives is preferably in the range 1-50 mg, or the administered single dose of deoxypeganine or one of its pharmacologically acceptable salts or derivatives is preferably in the range 10-500 mg, and the administered single dose of acamprosate or one of its pharmacologically acceptable salts or derivatives is preferably in the range 100-5 000 mg, or the administered single dose of memantine or one of its pharmacologically acceptable salts or derivatives is preferably in the range 1-50 mg, or the administered single dose of the modulator of metabotropic glutamate receptors is preferably in the range 0.1-100 mg.

5. Active ingredient combination according to any of the preceding claims claim 1, characterized in that it is in the form of a pharmaceutical form which has a depot effect.

6. Active ingredient combination according to any of the preceding claims claim 1, characterized in that it is in the form of a medicament to be administered orally.

7. Active ingredient combination according to any of the preceding claims claim 1, characterized in that it is in the form of a medicament to be administered parenterally.

8. Active ingredient combination according to claim 7, characterized in that it is in the form of a medicament to administered transdermally.

9. Use of an active ingredient combination according to any of claims 1 to 8 claim 1 for pharmacological addictive substance or intoxicant therapy, in particular the therapy of alcoholism.

10. Use of an active ingredient combination according to any of claims 1 to 3 for producing a pharmaceutical form for pharmacological addictive substance or intoxicant therapy, in particular the therapy of alcoholism.

11. Use according to claim 10, characterized in that the pharmaceutical form is produced in the form of a dosage form which has a depot effect.

12. Use according to claim 10 or 11, characterized in that the pharmaceutical form is produced in the form of an oral dosage form.

13. Use according to claim 10 or 11, characterized in that the pharmaceutical form is produced in the form of a parenteral dosage form.

14. Use according to claim 13, characterized in that the pharmaceutical form is produced in the form of a transdermal dosage form.

15. Use according to any of claims 11 to 14 claim 11, characterized in that the pharmaceutical form comprises a single dose for administration of galanthamine or one of its pharmacologically acceptable salts or derivatives is preferably in the range 1-50 mg, or a single dose for administration of deoxypeganine or one of its pharmacologically acceptable salts or derivatives is preferably in the range 10-500 mg, and a single dose for administration of acamprosate or one of its pharmacologically acceptable salts or derivatives is preferably in the range 100-5 000 mg, or a single dose for administration of memantine or one of its pharmacologically acceptable salts or derivatives is preferably in the range 1-50 mg, or a single dose for administration of the modulator of metabotropic glutamate receptors is preferably in the range 0.1-100 mg.

16. Method for pharmacological addictive substance or intoxicant therapy, in particular of alcoholism, characterized in that an active ingredient combination according to one or mere of claims 1 to 9 claim 1 is administered.
